(19) United States
(12) Patent Application Publication
(10) Pub. No.: US 2026/0232971 A1
(43) Pub. Date: Aug. 13, 2026

(54) ASSEMBLIES AND METHODS FOR INTRODUCING AN ADDITIONAL GUIDEWIRE

(71) Applicant: BLUE WIRE LTD, Haifa (IL)

(72) Inventors: Elias HELLOU, HAIFA (IL); Omri NAVEH, Alon Hagalil (IL)

(21) Appl. No.: 19/155,248

(22) PCT Filed: Feb. 22, 2024
(86) PCT No.: PCT/IB2024/051726

§ 371 Date: Aug. 10, 2025

Related U.S. Application Data
(60) Provisional application No. 63/447,904, filed on Feb. 24, 2023.

(51) Int. Cl. A61M 25/09; A61M 25/01

(52) U.S. Cl. CPC A61M 25/09041; A61M 2025/0177

(57) ABSTRACT

A medical system for coupling to a previously-introduced guidewire during an intracorporeal procedure comprises: a supplemental guide wire and a single-lumen tubular member comprising a radial wall fixedly engaged to a lengthwise portion of the supplemental guidewire The radial wall defines a passageway sized for slidable passage therethrough of the previously introduced guidewire. The tubular portion and the engaged lengthwise portion of the supplemental guidewire are not engaged coaxially.

### CROSS-REFERENCE TO RELATED APPLICATIONS

This patent application claims the benefit of U.S. provisional Patent Application No. 63/447,904 filed on Feb. 24, 2023 which is incorporated herein by reference in its entirety.

### FIELD OF THE INVENTION

Embodiments of the present invention relate generally to medical devices and more particularly to methods and apparatus for introducing a second guidewire to a previously-introduced guidewire in a lumen of a subject.

### BACKGROUND

Guidewires are used for navigating to reach a location within a patient such as a surgical or other treatment site. Once the tip of the guidewire arrives at its destination, it acts as a guide that larger elements, e.g., catheters, can rapidly follow for easier delivery to the treatment site. Introduction of an additional guidewire in parallel to a previously-introduced guidewire can be useful in many different procedures.

In order to ensure that the second guidewire follows the same path as the first, the introduction can be done using a coupling device. Such coupling tips have suffered from a number of design flaws. The most serious flaws include: the pathway provided for the previously-introduced guidewire through the coupling tip is not uniformly parallel to the second guidewire, or close enough to parallel; the pathway provided for the previously-introduced guidewire through the coupling tip is not at a fixed orientation to the second guidewire, i.e., the orientation can change when an external force is applied on the coupling device; the pathway provided for the previously-introduced guidewire through the coupling tip is not uniformly straight, i.e., the previously-introduced guidewire is forced to bend, often in more than one place, as the coupling device traverses the length of the guidewire; the pathway provided for the previously-introduced guidewire through the coupling tip is too short; and the passage provided in the coupling tip for the distal end of the second guidewire is not adaptable to the diameter of the guidewire. Any or all of these design flaws can mean that when introducing the second guidewire using such coupling tips, the surgeon can meet resistance and inadvertently cause shear forces on the previously-introduced guidewire, or even force uncouple the coupling device from the second guidewire.

Therefore, there is a need for a guidewire assembly that can be as effective as a coupling tip while eliminating the foregoing disadvantageous effects.

### SUMMARY OF THE INVENTION

According to embodiments disclosed herein, a medical system for coupling to a previously-introduced guidewire during an intracorporeal procedure comprises: (a) a supplemental guidewire; and (b) a single-lumen tubular member comprising a radial wall fixedly engaged to a lengthwise portion of the supplemental guidewire, the radial wall defining a passageway sized for slidable passage therethrough of the previously introduced guidewire, wherein the tubular portion and the engaged lengthwise portion of the supplemental guidewire are not engaged coaxially.

In some embodiments, it can be that the tubular portion and the supplemental guidewire are not coaxial. In some embodiments, a central longitudinal axis of the supplemental guidewire can be parallel to a central longitudinal axis of the tubular member. In some embodiments, a central longitudinal axis of the supplemental guidewire can be laterally displaced from a central longitudinal axis of the tubular member. In some embodiments, the lateral displacement is by at least 5% or at least 10% of the internal diameter of the tubular member. In some embodiments, the lateral displacement is by at least 15% or at least 20% of the internal diameter of the tubular member. In some embodiments, the lateral displacement is by at least 25% or at least 30% of the internal diameter of the tubular member. In some embodiments, the lateral displacement is by at least 35% or at least 40% of the internal diameter of the tubular member. In some embodiments, the lateral displacement is by at least 45% or at least 50% of the internal diameter of the tubular member. In some embodiments, the lateral displacement is more than 50% of the internal diameter of the tubular member. In some embodiments, a central longitudinal axis of the engaged lengthwise portion of the supplemental guidewire can be parallel to and laterally displaced from a central longitudinal axis of the tubular member.

In some embodiments, the engaged lengthwise portion of the supplemental guidewire can be at least partly embedded longitudinally in the radial wall of the tubular member.

In some embodiments, the fixed engaging of the tubular member with the lengthwise portion can include at least one of molding, overmolding, extruding, welding, heat-welding, gluing, soldering, and fusing. In some embodiments, the tubular member can be molded and/or extruded to at least partly embed, in said radial wall, the lengthwise portion of the supplemental guidewire.

In some embodiments, an angular majority of a circumference of the engaged lengthwise portion can be in fixed contact with the radial wall. In some embodiments, it can be that at most an angular minority of a circumference of the engaged lengthwise portion is in fixed contact with the radial wall. In some embodiments, it can be that the engaged lengthwise portion of the supplemental guidewire includes a distal end of the supplemental guidewire. In some embodiments, it can be that the engaged lengthwise portion of the supplemental guidewire is not indestructible separable from the tubular member.

A method is disclosed, according to embodiments, for using multiple guidewires. The method comprises: (a) providing the medical system according to any of the foregoing embodiments of the medical system; (b) locating a proximal end of the previously-introduced guidewire; (c) slidably passing the tubular member of the medical system over the proximal end of the previously-introduced guidewire so that said proximal end enters a distal opening of the tubular member and exits a proximal opening thereof; and (d) distally advancing the medical system over the previously-introduced guidewire.

A method is disclosed, according to embodiments, for coupling to a previously-introduced guidewire during an intracorporeal procedure, the method comprising: (a) providing a medical system comprising a supplemental guidewire and a single-lumen tubular member comprising a radial wall fixedly and non-coaxially engaged with a lengthwise portion of the supplemental guidewire; (b) locating a proximal end of a previously-introduced guidewire; (c) slidably passing the tubular member of the medical system over the proximal end of the previously-introduced guidewire so that said proximal end enters a distal opening of the tubular member and exits a proximal opening thereof; and (d) distally advancing the medical system over the previously-introduced guidewire.

In some embodiments of the method, it can be that the tubular portion and the supplemental guidewire are not coaxial. In some embodiments, a central longitudinal axis of the supplemental guidewire can be parallel to a central longitudinal axis of the tubular member. In some embodiments, a central longitudinal axis of the supplemental guidewire can be laterally displaced from a central longitudinal axis of the tubular member. In some embodiments, a central longitudinal axis of the engaged lengthwise portion of the supplemental guidewire can be parallel to and laterally displaced from a central longitudinal axis of the tubular member.

In some embodiments, the engaged lengthwise portion of the supplemental guidewire can be at least partly embedded longitudinally in a radial wall of the tubular member. In some embodiments, the tubular member can be molded and/or extruded to at least partly embed, in said radial wall, the lengthwise portion of the supplemental guidewire.

In some embodiments, an angular majority of a circumference of the lengthwise portion can be in fixed contact with the radial wall. In some embodiments, the engaged lengthwise portion of the supplemental guidewire can include a distal end of the supplemental guidewire. In some embodiments, it can be that the engaged lengthwise portion of the supplemental guidewire is not indestructible separable from the tubular member.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1A shows a schematic partial side view of a guidewire assembly according to embodiments of the present invention.

FIGS. 1B and 1C show, schematically, various non-limiting examples of transverse cross-sections of guidewire assemblies, according to embodiments of the present invention.

FIG. 2A shows a schematic partial side view of a second guidewire according to embodiments of the present invention.

FIG. 2B shows illustrates additional features of the guidewire assembly of FIG. 1A.

FIG. 2C shows the second guidewire of FIG. 2A passing through the tubular member of the guidewire assembly of FIGS. 1A and 2B, according to embodiments of the present invention.

FIGS. 3A, 3B, 4, 5, 6A, 6B and 7A show schematic partial side views of guidewire assemblies according to embodiments of the present invention.

FIG. 7B shows a schematic top view of the guidewire used the assembly of FIG. 7A according to embodiments of the present invention.

FIGS. 8A and 8B show schematic partial side views of guidewire assemblies according to embodiments of the present invention.

FIG. 8C shows a schematic top view of the guidewire assembly of FIG. 8A according to embodiments of the present invention.

FIG. 8D shows a schematic side view of the guidewire assembly of FIG. 8A with a second guidewire passing through the tubular member of the guidewire assembly according to embodiments of the present invention.

FIG. 9A shows a schematic side view of a guidewire for use in a guidewire assembly according to embodiments of the present invention.

FIGS. 9B and 9C show respective schematic side views of a guidewire assembly including the guidewire of FIG. 9A according to embodiments of the present invention.

FIG. 9D shows a schematic top view of a guidewire assembly including the guidewire of FIG. 9A according to embodiments of the present invention.

FIG. 10 shows a schematic side view of the guidewire assembly of FIGS. 9B and 9C with a second guidewire passing through the tubular member of the guidewire assembly according to embodiments of the present invention.

FIGS. 11 and 12 show respective flowcharts of methods for using multiple guidewires according to embodiments of the present invention.

FIGS. 13A, 13B, 13C and 13D illustrate steps of a method for coupling a guidewire assembly to a previously introduced second guidewire during a medical procedure, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

As used herein and in the appended claims and figures, the terms ‘guidewire assembly’ and ‘medical system’ are equivalent and interchangeable. As used herein and in the appended claims and figures, the terms ‘first guidewire’ and ‘second guidewire’ mean, respectively, a guidewire incorporated in a guidewire assembly/medical system according to the disclosed embodiments, and a guidewire previously introduced into a cavity or lumen. The term ‘supplemental guidewire’ is equivalent to and interchangeable with ‘first guidewire’. The term ‘previously-introduced guidewire’ is equivalent to and interchangeable with ‘second guidewire’. The term ‘engaged with’ means attached to, affixed to, integrated with, and the like.

The terms ‘distal’ and ‘proximal’ are used in this disclosure and the appended claims as follows: ‘distal’ means further into the body (along an insertion path) from a point of entry into the body, while ‘proximal’ means closer to the point of entry into the body. Where the terms are used in reference to apparatus outside of a patient's body, a distal portion or distal end can be that portion or end of the apparatus configured to be inserted into the body first, such as the tubular member of a guidewire assembly, while a proximal end or proximal portion is either inserted last (or may never be inserted). Additionally or alternatively, when the terms are used in reference to apparatus outside of a patient's body, a distal portion or distal end can be that portion farther or farthest from a user grasping the apparatus, whilst the corresponding proximal portion or proximal can be that portion closer or closest to the user grasping the apparatus. Additionally, when used relatively, e.g. ‘distally displaced from’ or ‘proximal of’, the meaning is, respectively, closer to the distal end than’ or ‘closer to the proximal end than’. The respective distal and proximal directions are shown in many of the figures, and in all relevant figures the convention of ‘distal to the left’ and ‘proximal to the right’ is maintained.

The terms “first” and “second” with respect to guidewires and respective portions of coupling devices are used throughout this disclosure and in the appended claims in accordance with the following convention: The “first guidewire” means the guidewire that is part of a guidewire assembly, which a surgeon may couple to a guidewire previously introduced in a lumen of a patient. Thus, the previously-introduced guidewire is referred to as the “second guidewire”.

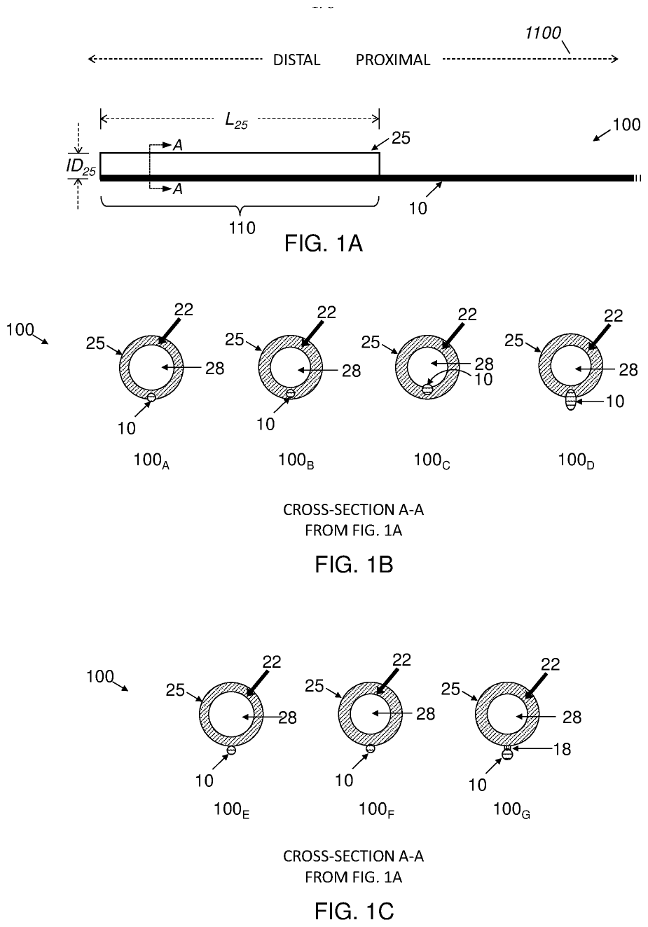

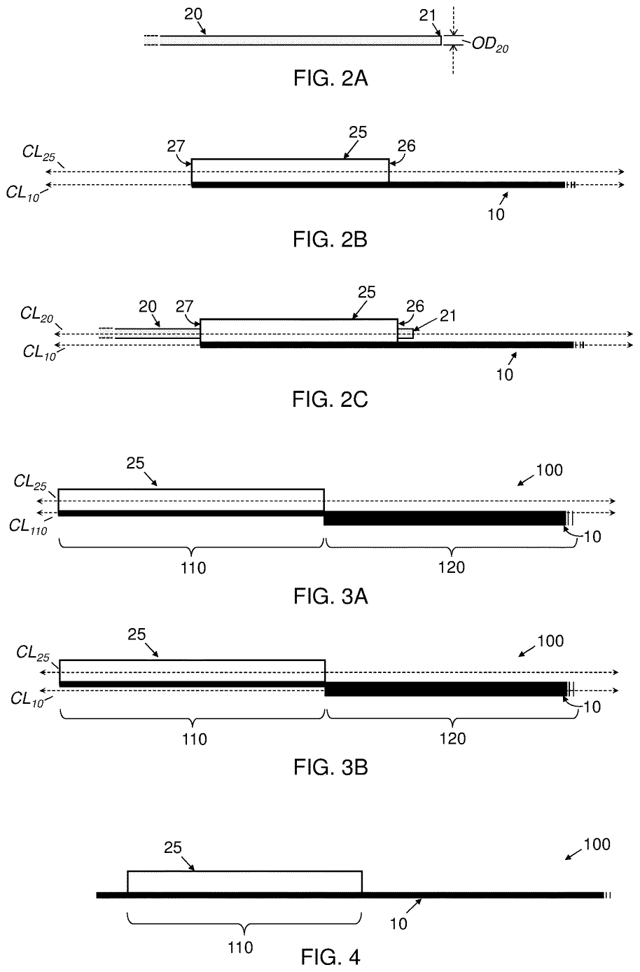

Referring now to the figures and in particular to FIGS. 1A, 1B and 1C, a guidewire assembly 100 includes a first guidewire 10 and a tubular member 25. The guidewire assembly 100 is provided for coupling the first guidewire 10 to a second guidewire, e.g., second guidewire 20 of FIGS. 2A-B, which has already been introduced to a lumen. The tubular member 25 and the second guidewire are selected so that the second guidewire is slidably passable through the interior of the tubular member 25.

The non-limiting example of a guidewire assembly 100 illustrated schematically in FIG. 1A shows the tubular member 25 fixedly engaged with a distal portion 110 of the first guidewire 10. Respective distal and proximal directions are shown by the arrow 1000 in FIG. 1A; for convenience, the convention of distal=left and proximal=right is maintained through FIGS. 2A, 2B, 3, 4, and 5. The tubular member 25 and guidewire 10 of FIG. 1A cannot be non-destructively separated or detached. Examples of attachment methods within the definition of ‘fixedly engaged’ include, and not exhaustively: molding, overmolding, extruding, welding, heat-welding, gluing, soldering, and fusing.

In embodiments, a tubular member 25 may be formed from a flexible or elastic material, such as a polymer or a metal (or metal alloy), or from a super-elastic material, such as a nitinol. An attachment method for the fixed engaging can be selected in accordance with the material selected; alternatively, the material of the tubular member 25 can be selected in accordance with a preferred method of attachment.

The tubular member 25 is meant to allow the guidewire assembly 100 to slide along the second guidewire (the previously-introduced guidewire) is sized to be long enough that it is not particularly difficult to slide the guidewire assembly 100 without damaging the second guidewire or getting stuck. Accordingly, the tubular section 25 is provided, according to embodiments, with both a minimum length and a minimum length-to-diameter ratio that in combination equip the guidewire assembly 100 with an enhanced ability to traverse the second guidewire safely and effectively. The tubular member 25 has a length indicated in FIG. 1A by the arrow marked L₂₅, and an inner diameter marked ID₂₅. In embodiments, the length L₂₅ is suitably at least 1 cm. In some embodiments, the length L₂₅ is at least 2 cm. In other embodiments, the length L₂₅ is at least 3 cm. In yet other embodiments, the length L₂₅ is at least 4 cm. In some embodiments, the length L₂₅ is between 1-2 cm, or 1-3 cm, or 1-4 cm, or 1-5 cm, or 2-3 cm, or 2-4 cm, or 2-5 cm, or 3-4 cm, or 3-5 cm, or longer. All ranges cited in this disclosure (and in the appended claims) is an inclusive range.

In embodiments, the inner diameter ID₂₅ is at least 0.95 mm and no more than 2 mm. In some embodiments, the inner diameter ID₂₅ is at least 0.95 mm and no more than 3 mm. In some other embodiments, the inner diameter ID₂₅ is at least 0.5 mm and no more than 2 mm. In some other embodiments, the inner diameter ID₂₅ is at least 0.5 mm and no more than 1.5 mm. In yet other embodiments, the inner diameter ID₂₅ is at least 0.5 mm and no more than 3 mm. In still other embodiments, the inner diameter ID₂₅ is at least 0.2 mm and no more than 2 mm. In yet other embodiments, the inner diameter ID₂₅ is at least 0.2 mm and no more than 1 mm. In still other embodiments, the inner diameter ID₂₅ is at least 0.2 mm and no more than 3 mm.

The ratio of the length L₂₅ to the inner diameter ID₂₅ is between 100:1 and 5:1, or between 50:1 and 5:1, or between 30:1 and 5:1, or between 100:1 and 10:1, or between 50:1 and 10:1, or between 30:1 and 10:1, or between 100:1 and 30:1, or between 50:1 and 30:1.

FIG. 1B shows exemplary cross-sectional views of a guidewire assembly 100 according to various embodiments in which the engaged distal portion 110 of the guidewire 10 is at least partly embedded longitudinally in a radial wall 22 of the tubular member 25. The radial wall 22 defines a passageway 28 sized for slidable passage therethrough of a guidewire (not shown in FIG. 1B), e.g., a guidewire previously introduced into a cavity or lumen. The embedding can be accomplished, for example, by molding, i.e., overmolding, or extruding the radial wall 22 of the tubular member 25 over the distal portion 110 of the guidewire 10, using a thermosetting polymer. In each of the first three examples shown in FIG. 1B as respective cross-sections of guidewire assemblies 100_{A}, 100_{B}, 100_{C}, the engaged lengthwise portion of the first guidewire has a diameter less than a thickness of the radial wall 22. For guidewire assemblies 100_{A}, 100_{B}, 100_{C}, it can be seen that an angular majority of a circumference of the engaged lengthwise portion 110 of guidewire 10 is in fixed contact with the radial wall 22. In some designs, the angular majority is at least 70% of the circumference of the engaged lengthwise portion 110 that is in fixed contact with the radial wall 22, and in some designs at least 90%. The guidewire assembly 100_{D} of FIG. 1B differs from the other examples in that the guidewire 10 has a maximum dimension greater than the thickness of the radial wall 22 of the tubular member 25, and an angular minority of the guidewire 10 is in contact with the material of the radial wall 22. Guidewire assembly 100_{D} also illustrates that a guidewire can have a cross-section that is not circular.

FIG. 1C shows exemplary cross-sectional views of a guidewire assembly 100 according to various embodiments in which the fixedly engaged distal portion 110 of the guidewire 10 is attached externally, or at least mostly externally, to the radial wall 22 of the tubular member 25. The attachment can be accomplished, for example, by welding, heat-welding, soldering, fusing, or gluing the distal portion 110 of the guidewire 10 to the tubular member 25. Exemplary cross-sections of guidewire assemblies 100_{E}, 100_{F}, and 100_{G} illustrate the extent to which the guidewire 10 contacts the tubular member 25 in such configurations—at most an angular minority as seen in guidewire assemblies 100_{E}, 100_{F}, including without direct contact with the tubular member 25, e.g., only through an intervening material 18 such as glue or solder. Thus, the expression “at most an angular minority” means ‘either an angular minority or no direct contact at all’.

FIG. 2A shows a typical guidewire, for use with the guidewire assembly 100 of the various embodiments disclosed herein, as a second guidewire 20, and specifically as a guidewire that is deployed by a surgeon before using the guidewire assembly 100. The second guidewire 20 includes a proximal end 21 which remains outside of the patient's body at all times. The second guidewire 20 has an outer diameter of OD₂₀. The inner diameter ID₂₅ of the tubular member 25 of the guidewire assembly 100, as shown in FIG. 1A, is selected to be at least slightly bigger than the outer diameter OD₂₀ of the second guidewire 20.

FIG. 2B illustrates additional features of the guidewire assembly 100 of FIGS. 1A and 2B. The tubular member 25 comprises respective distal and proximal opening 27, 26. A centerline of the tubular member 25, indicated by arrow CL₂₅ and passing through respective distal and proximal openings 27, 26, can be seen to be parallel, within manufacturing tolerances (of the guidewire assembly 100) of ±1° or ±2° from parallel, to a centerline of the first guidewire 10, indicated by arrow CL₁₀. As shown, e.g., in FIGS. 2B, 3B, 6A, and 9C, in any of the embodiments disclosed herein the centerline CL₂₅ of the tubular member 25 can be laterally (or, equivalently, radially displaced, from the centerline CL₁₀ of the first guidewire 10. In some embodiments, the lateral displacement is by at least 5% or at least 10% of the internal diameter ID₂₅ of the tubular member. In some embodiments, the lateral displacement is by at least 15% or at least 20% of the internal diameter ID₂₅ of the tubular member 25. In some embodiments, the lateral displacement is by at least 25% or at least 30% of the internal diameter ID₂₅ of the tubular member 25. In some embodiments, the lateral displacement is by at least 35% or at least 40% of the internal diameter ID₂₅ of the tubular member 25. In some embodiments, the lateral displacement is by at least 45% or at least 50% of the internal diameter ID₂₅ of the tubular member 25. In some embodiments, the lateral displacement is more than 50% of the internal diameter ID₂₅ of the tubular member 25, e.g., as can be understood from the examples of FIG. 1C.

FIG. 2C illustrates the second guidewire 20 passing through the tubular member 25 of the guidewire assembly 100. The proximal end 21 of the second guidewire 20 passes through the distal opening 27 of the tubular member 25 and then through the proximal opening 26. Then the remainder of the second guidewire 20 passes through the inner lumen of the tubular member 25 as the guidewire assembly slides over and along the second guidewire 20. Whether the proximal end 21 of the second guidewire 20 passes through the openings 27, 26 and through the tubular member 25, or the tubular member 25 passes over the proximal end 21, is merely a matter of perspective and the two actions, or two descriptions, are equivalent. A centerline of the second guidewire 20, indicated by arrow CL₂₀, can be seen to be parallel, within manufacturing tolerances (of the guidewire assembly 100) of ±1° or ±2° from parallel, to a centerline of the first guidewire 10, indicated by arrow CL₁₀.

Referring now to FIGS. 3A-B, 4, 5 and 6A-B, various aspects of fixed engagement between the guidewire 10 and the tubular member 25 of the guidewire assembly 100 are shown.

FIGS. 3A and 3B show a partial schematic side view of a guidewire assembly 100 in which the engaged portion 110 of the guidewire 10, i.e., the part fixedly engaged with the tubular member 25, is thinner than a different portion 120 of the guidewire 10 that is not engaged with the tubular member 25. In some examples (not shown), the engaged portion 110 is thicker than the different (unengaged) portion 120. As shown in FIG. 3A, a centerline of the tubular member 25, indicated by arrow CL₂₅ and passing through respective distal and proximal openings 27, 26, can be seen to be parallel, within manufacturing tolerances (of the guidewire assembly 100) of ±1° or ±2° from parallel, to a centerline of the engaged portion 110 of the first guidewire 10, as indicated by arrow CL₁₁₀. As shown in FIG. 3B, a centerline of the tubular member 25, indicated by arrow CL₂₅ and passing through respective distal and proximal openings 27, 26, can be seen to be parallel, within manufacturing tolerances (of the guidewire assembly 100) of ±1° or +2° from parallel, to a centerline of the first guidewire 10, indicated by arrow CL₁₀.

FIG. 4 shows a partial schematic side view of a guidewire assembly 100 in which the engaged lengthwise portion 110 of the first guidewire 10 does not include a distal end of the guidewire 10, in contrast, e.g., to FIGS. 1A, 2B and 3A-B.

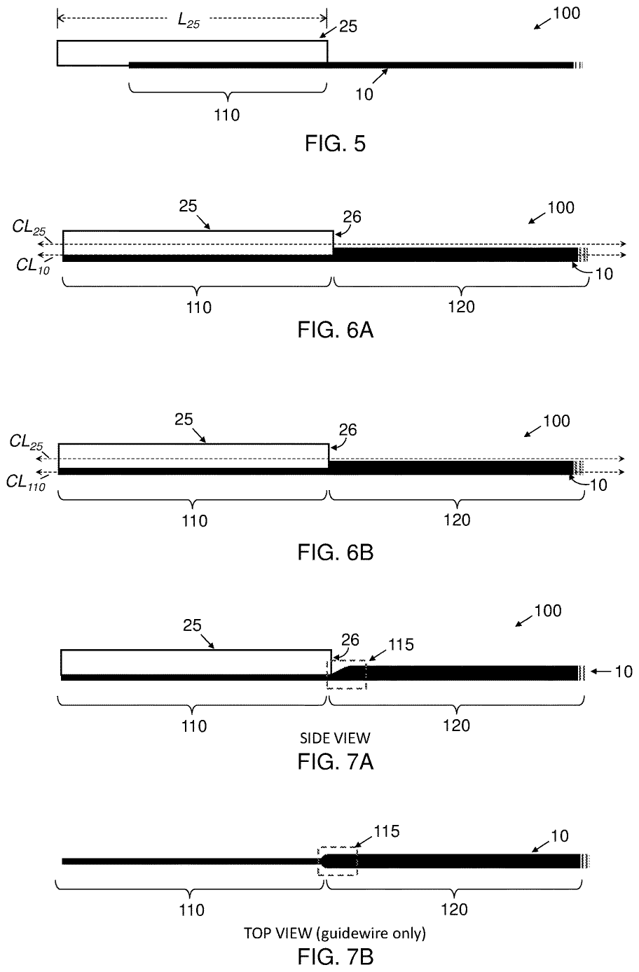

FIG. 5 shows a partial schematic side view of a guidewire assembly 100 in which the engaged lengthwise portion 110 of the guidewire 10 is engaged with at least a lengthwise majority of the tubular member 25; in other words, the length of the engaged portion 110 is at least half of length L₂₅ of the tubular member 25. In contrast, as shown, for example, in FIGS. 1A, 2B, and 3A-B, the engaged lengthwise portion 110 of the guidewire 10 can be engaged with substantially all of the tubular member 25, where ‘substantially’means at least 95% or at least 97% or at least 99%.

FIGS. 6A and 6B show a partial schematic side view of a guidewire assembly 100 in which the engaged portion 110 of the guidewire 10, i.e., the part fixedly engaged with the tubular member 25, is thinner than a different portion 120 of the guidewire 10 that is not engaged with the tubular member 25. In some examples (not shown), the engaged portion 110 is thicker than the different (unengaged) portion 120. The exemplary guidewire assembly 100 of FIGS. 6A-B is superficially similar to that of FIGS. 3A-B; in FIGS. 6A-B, the thicker proximal portion 120 of the first guidewire 10 is arranged so that its central longitudinal axis intersects the inner lumen of the tubular member 25 at the proximal opening 26, wherein in FIGS. 3A-B it did not. As shown in FIG. 6A, a centerline of the tubular member 25, indicated by arrow CL₂₅ and passing through respective distal and proximal openings 27, 26, can be seen to be parallel, within manufacturing tolerances (of the guidewire assembly 100) of ±1° or ±2° from parallel, to a centerline of the first guidewire 10, indicated by arrow CL₁₀. As shown in FIG. 6B, a centerline of the tubular member 25, indicated by arrow CL₂₅ and passing through respective distal and proximal openings 27, 26, can be seen to be parallel, within manufacturing tolerances (of the guidewire assembly 100) of ±1° or +2° from parallel, to a centerline of the engaged portion 110 of the first guidewire 10, as indicated by arrow CL₁₁₀.

We now refer to FIGS. 7A-B and 8A-D, in which the first guidewire 10 includes a transition portion 115 mediating between the proximal portion 120 and the engaged lengthwise portion 110. Inter alia, the transition portion adds structural robustness to the meeting between the thicker proximal portion 120 and thinner engaged portion 110. In the non-limiting example of FIG. 7A, the transition portion 115 is located proximal to the proximal opening 26 of the tubular member 25. The ‘slope’ of the transition portion 115 as seen in the side view of FIG. 7A is shown as a non-limiting example, and the shape of the transition portion 115 is a matter of design choice. Similarly, the footprint of the transition portion 115 as shown in the top view of FIG. 7B represents a specific design choice. In other examples, the three-dimensional shape of the transition portion 115 may take into account the forces acting upon this portion of the guidewire 10 during deployment.

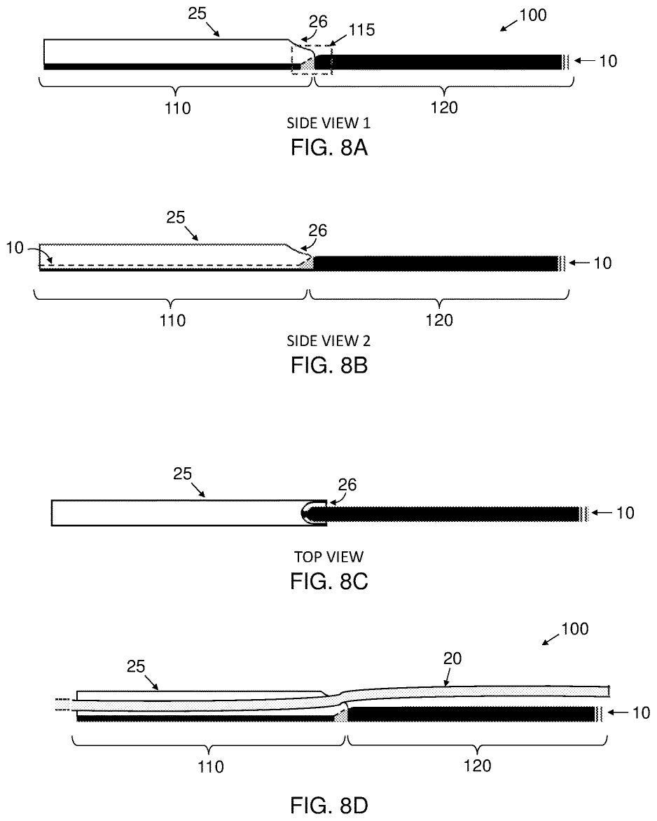

The guidewire assembly 100 of FIG. 8A is superficially similar to that of FIG. 7A. the guidewire assembly 100 of FIG. 8A is characterized by at least part of the transition portion 115 overlapping longitudinally with the proximal end of the tubular member 25 to be inside the interior lumen of the tubular member 25. Additionally or alternatively, the proximal opening 26 of the tubular member 25 in FIG. 8A has a scalloped or cut-out edge opposite, i.e., diametrically opposed to, the position of the first guidewire 10. The shape of the edge is intended, inter alia, to facilitate the slidable passage of the tubular member 25 around a second guidewire 20. In embodiments, the guidewire assembly 100 is formed to allow slidable passage of the tubular portion 25 over a second guidewire 20 having a maximum diameter that is equal to a maximum diameter of the first guidewire 10. FIG. 8B schematically illustrates an alternate design to the guidewire assembly of FIG. 8A. In FIG. 8A, an angular majority of a circumference of the engaged lengthwise portion 110 is disposed outside the tubular member 25 and/or the engaged lengthwise portion 110 does not impinge on the interior lumen of the tubular member 25. In FIG. 8B, an angular majority of a circumference of the engaged lengthwise portion 110 is disposed inside the tubular member 25.

FIG. 8C shows a top view of a guidewire assembly 100, e.g., the guidewire assembly of FIG. 8A, showing a non-limiting example of a shape of the cut-out of the proximal edge of the tubular member 25 at the proximal opening 26.

FIG. 8D is a schematic illustration of the proximal end of a second guidewire 20 passing through the tubular portion 25 of the guidewire assembly 100 of FIGS. 8A and 8C. In some embodiments, the second guidewire 20 remains parallel, or can remain parallel, to the first guidewire 10 along the length of any overlap. In some embodiments, the second guidewire 20 remains parallel, or can remain parallel, to the first guidewire 10 along the length of any overlap with the except of a slight turn at the proximal opening 26 of the tubular member 25.

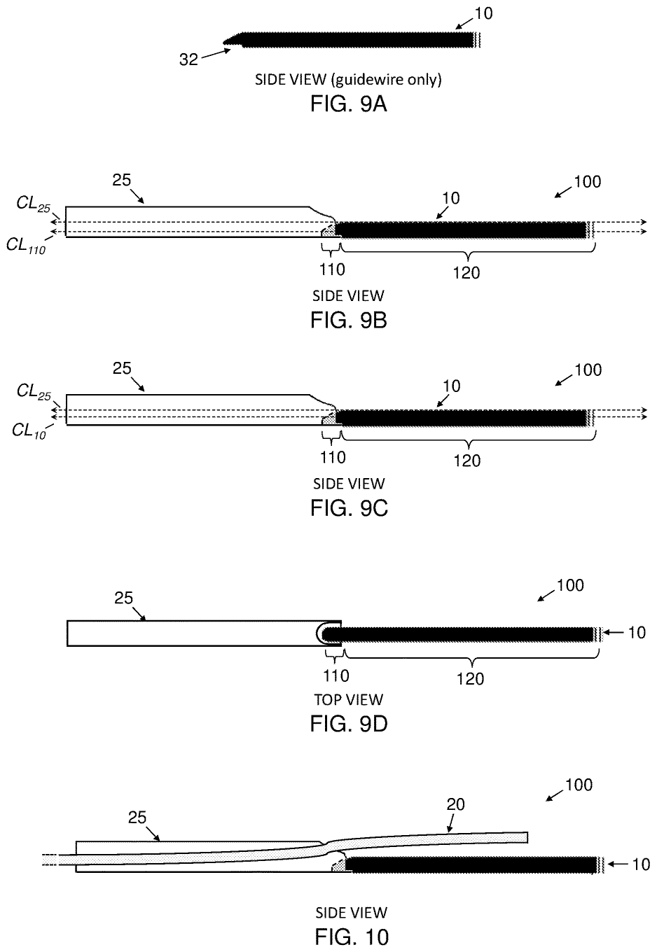

We now refer to FIGS. 9A-D and 10. A guidewire assembly 100 comprises a first guidewire 10 and a tubular member 25. The tubular member 25 is fixedly and non-coaxially engaged with a lengthwise portion 110 of the first guidewire 10. The exemplary first guidewire 10 of FIGS. 9A-D and 10 includes an engaged lengthwise portion 110 similar to the transition portion 115 of FIGS. 7A-B and 8A-D, but does not include a lengthwise portion further elongated distally therefrom. As shown in FIG. 9B, a centerline of the tubular member 25, indicated by arrow CL₂₅ and passing through respective distal and proximal openings 27, 26, can be seen to be parallel, within manufacturing tolerances (of the guidewire assembly 100) of ±1° or ±2° from parallel, to a centerline of the engaged portion 110 of the first guidewire 10, as indicated by arrow CL₁₁₀. As shown in FIG. 9C, a centerline of the tubular member 25, indicated by arrow CL₂₅ and passing through respective distal and proximal openings 27, 26, can be seen to be parallel, within manufacturing tolerances (of the guidewire assembly 100) of ±1° or ±2° from parallel, to a centerline of the first guidewire 10, indicated by arrow CL₁₀. As shown in FIG. 6B, a centerline of the tubular member 25, indicated by arrow CL₂₅ and passing through respective distal and proximal openings 27, 26, can be seen to be parallel, within manufacturing tolerances (of the guidewire assembly 100) of ±1° or +2° from parallel, to a centerline of the engaged portion 110 of the first guidewire 10, as indicated by arrow CL₁₁₀.

As can be seen in FIG. 10, the guidewire assembly 100 is formed to allow slidable passage of the tubular portion 25 over a second guidewire 20. While the second guidewire 20 is not illustrated in the schematic drawing of FIG. 10, the guidewire assembly 100 is formed to allow slidable passage of the tubular portion 25 over a second guidewire 20 having a maximum diameter that is equal to a maximum diameter of the first guidewire 10.

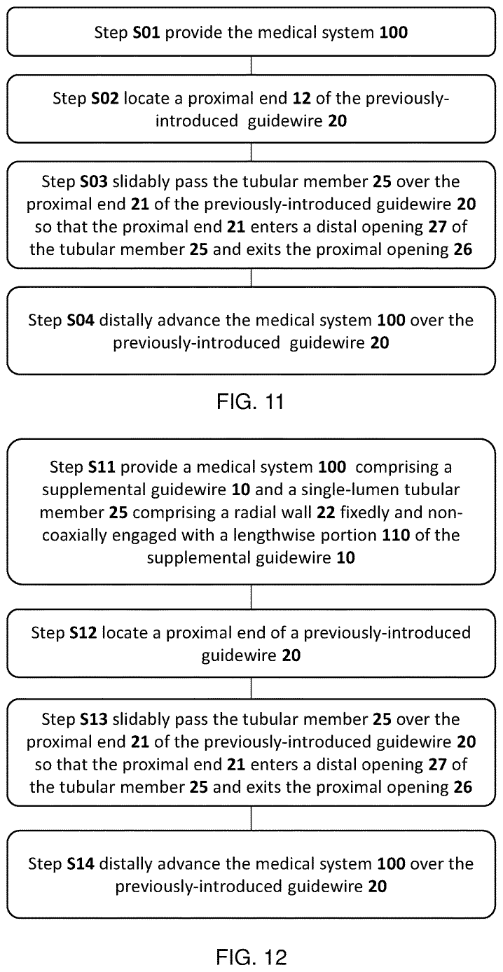

Referring now to FIG. 11, a method is disclosed for using multiple guidewires using a guidewire assembly 100, for example, any of the guidewire assemblies 100 disclosed hereinabove and/or illustrated in FIGS. 1A, 2B, 3-6, 7A-B, 8A-8B, 8D, 9B-D and 10. For example, using the multiple guidewires can include coupling a first guidewire 10 to a previously introduced second guidewire 20 during a medical procedure.

As illustrated by the flow chart in FIG. 11, the method comprises at least 4 method steps S01, S02, S03, and S04.
Step S01 includes providing the medical system 100, i.e., according to any one of the embodiments disclosed herein, and/or illustrated in FIGS. 1A, 2B, 3-6B, 7A-B, 8A-8B, 8D, 9B-D and 10.
Step S02 includes locating a proximal end 21 of the previously-introduced guidewire 20.
Step S03 includes slidably passing the tubular member 25 of the medical system 100 over the proximal end 21 of the previously-introduced guidewire 20. The proximal end 21 enters a distal opening 27 of the tubular member 25 and exits the proximal opening 26.
Step S04 includes distally advancing the medical system 100 over the previously-introduced guidewire 20.

In some embodiments, performance of Step S04 is optional.

Referring now to FIG. 12, a method is disclosed for using multiple guidewires using a medical system 100. For example, using the multiple guidewires can include coupling a first guidewire 10 to a previously introduced second guidewire 20 during a medical procedure.

As illustrated by the flow chart in FIG. 12, the method comprises at least 4 method steps S11, S12, S13, and S14.
Step S01 includes. providing a medical system 100 comprising a supplemental guidewire 10 and a single-lumen tubular member 25 comprising a radial wall 22 fixedly and non-coaxially engaged with a lengthwise portion 110 of the supplemental guidewire 10.
Step S12 includes locating a proximal end 21 of the second guidewire 20.
Step S13 includes slidably passing the tubular member 25 of the guidewire assembly 100 over the proximal end 21 of the previously-introduced guidewire 20. The proximal end 21 enters a distal opening 27 of the tubular member and exits the proximal opening 26.
Step S14 includes distally advancing the medical system 100 over the previously-introduced guidewire 20.

In some embodiments, performance of Step S14 is optional.

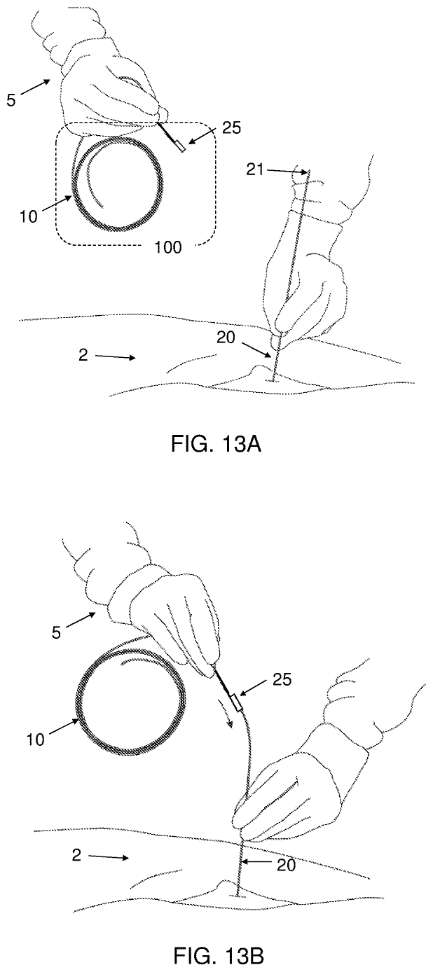

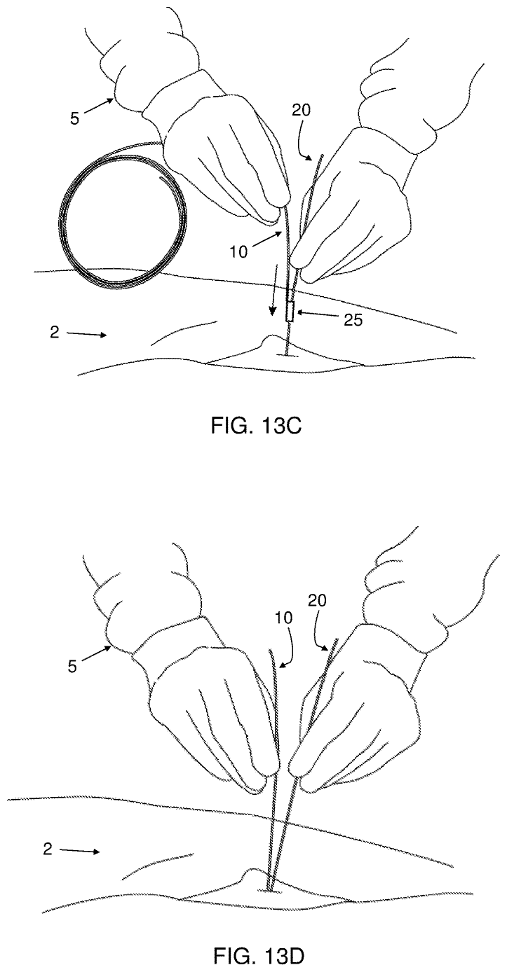

We now refer to FIGS. 13A-13D, which illustrate a method for using multiple coupling a first guidewire to a previously introduced second guidewire during a medical procedure.

In a first method step, a guidewire assembly 100 according to any of the embodiments disclosed hereinabove, comprising a tubular member 25 in accordance with any of the embodiments disclosed herein and a first guidewire 10 fixedly engaged with the tubular member 25, is provided. FIG. 13A shows a surgeon 5 holding a proximal end 21 of a ‘second’ guidewire 20 previously introduced in a lumen of patient 2. The surgeon 5 also holds the first guidewire 10 of the provided apparatus 100.

A second method step is illustrated in FIG. 13B, wherein the surgeon 5 slidably passes the proximal end 21 of the second guidewire 20 through the tubular member 25 of the guidewire assembly 100 in a straight-line path between respective distal and proximal openings 27, 26 of the tubular member 25. The straight-line path has a length-to-diameter ratio of at least 5:1, or at least 10:1.

FIGS. 13C and 13D illustrate additional method steps that are optional in some embodiments. In FIG. 13C, the surgeon 5 is shown sliding the tubular member 25 of the guidewire assembly 100 distally along the second guidewire 20 in preparation for advancing the first guidewire 10 into the lumen or vessel of the patient 2. FIG. 13D shows the surgeon 5 holding respective proximal ends of the first and second guidewires 10, 20, both of which have been introduced into the lumen or vessel of the patient 2, the ‘first’ guidewire 10 introduced using the using the features of the guidewire assembly 100 and the associated methods disclosed in the embodiments.

### Inventive Concepts

Any of the disclosed embodiments may include, inter alia, any of the following inventive concepts, numbered 1-29 for convenience. Any one or more of the inventive concepts can be combined with any other embodiments of the current disclosure, within the scope of the invention.

Inventive Concept 1. A guidewire assembly comprising: (a) a first guidewire for use in a medical procedure; and (b) a tubular member fixedly engaged with a lengthwise portion of the first guidewire such that the engaged lengthwise portion of the first guidewire is parallel to a central longitudinal axis of the tubular member, the guidewire assembly being formed to allow slidable passage of the tubular member around a second guidewire.

Inventive Concept 2. The guidewire assembly of Inventive Concept 1, wherein the engaged lengthwise portion of the first guidewire is at least partly embedded longitudinally in a radial wall of the tubular member.

Inventive Concept 3. The guidewire assembly of Inventive Concept 2, wherein the engaged lengthwise portion of the first guidewire has a diameter less than a thickness of the radial wall.

Inventive Concept 4. The guidewire assembly of any one of the preceding Inventive Concepts, wherein the tubular member is molded and/or extruded to at least partly embed, in said radial wall, the lengthwise portion of the first guidewire.

Inventive Concept 5. The guidewire assembly of any one of the preceding Inventive Concepts, wherein the radial wall comprises a thermosetting polymer.

Inventive Concept 6. The guidewire assembly of any one of Inventive Concepts 2 to 5, wherein an angular majority of a circumference of the lengthwise portion is in fixed contact with the radial wall.

Inventive Concept 7. The guidewire assembly of any one of Inventive Concepts 2 to 6, wherein at least 70% of the circumference of the engaged lengthwise portion is in fixed contact with the radial wall.

Inventive Concept 8. The guidewire assembly of any one of Inventive Concepts 2 to 7, wherein at least 90% of the circumference of the engaged lengthwise portion is in fixed contact with the radial wall.

Inventive Concept 9. The guidewire assembly of Inventive Concept 1, wherein the engaged lengthwise portion of the first guidewire is at least one of welded, soldered, fused, and heat-welded to a radial wall of the tubular member such that no more than an angular minority of a circumference of the lengthwise portion is in fixed contact with the radial wall.

Inventive Concept 10. The guidewire assembly of any one of the preceding Inventive Concepts, wherein the engaged lengthwise portion of the first guidewire has a diameter less than a diameter of a different portion of the first guidewire that is not engaged with the tubular member.

Inventive Concept 11. The guidewire assembly of any one of the preceding Inventive Concepts, wherein the engaged lengthwise portion of the first guidewire includes a distal end of the first guidewire.

Inventive Concept 12. The guidewire assembly of any one of the preceding Inventive Concepts, wherein the engaged lengthwise portion of the first guidewire is engaged with at least a lengthwise majority of the tubular member.

Inventive Concept 13. The guidewire assembly of Inventive Concept 12, wherein the engaged lengthwise portion of the first guidewire is engaged with substantially all of the tubular member.

Inventive Concept 14. The guidewire assembly of any one of the preceding Inventive Concepts, wherein the engaged lengthwise portion of the first guidewire is not indestructible separable from the tubular member.

Inventive Concept 15. A method for using multiple guidewires, the method comprising: (a) providing the guidewire assembly of any one of the preceding Inventive Concepts; (b) locating a proximal end of the second guidewire; (c) slidably passing the tubular member of the guidewire assembly over the proximal end of the second guidewire so that said proximal end enters a distal opening of the tubular member and exits a proximal opening thereof; and (d) distally advancing the guidewire assembly over the second guidewire, the first guidewire being substantially parallel to the second guidewire.

Inventive Concept 16. A method for using multiple guidewires, the method comprising: (a) providing a guidewire assembly comprising a first guidewire and a tubular member fixedly engaged with a lengthwise portion of the first guidewire such that the engaged lengthwise portion of the first guidewire is parallel to a central longitudinal axis of the tubular member; (b) locating a proximal end of a second guidewire; (c) slidably passing the tubular member of the guidewire assembly over the proximal end of the second guidewire so that said proximal end enters a distal opening of the tubular member and exits a proximal opening thereof; and (d) distally advancing the guidewire assembly over the second guidewire, the first guidewire being substantially parallel to the second guidewire.

Inventive Concept 17. The method of Inventive Concept 16, wherein the engaged lengthwise portion of the first guidewire is at least partly embedded longitudinally in a radial wall of the tubular member.

Inventive Concept 18. The method of Inventive Concept 17, wherein the engaged lengthwise portion of the first guidewire has a diameter less than a thickness of the radial wall.

Inventive Concept 19. The method of any one of Inventive Concepts 16 to 18, wherein the tubular member is molded and/or extruded to at least partly embed, in said radial wall, the lengthwise portion of the first guidewire.

Inventive Concept 20. The method of any one of Inventive Concepts 16 to 19, wherein the radial wall comprises a thermosetting polymer.

Inventive Concept 21. The method of any one of Inventive Concepts 17 to 20, wherein an angular majority of a circumference of the lengthwise portion is in fixed contact with the radial wall.

Inventive Concept 22. The method of any one of Inventive Concepts 17 to 21, wherein at least 70% of the circumference of the engaged lengthwise portion is in fixed contact with the radial wall.

Inventive Concept 23. The method of any one of Inventive Concepts 17 to 22, wherein at least 90% of the circumference of the engaged lengthwise portion is in fixed contact with the radial wall.

Inventive Concept 24. The method of Inventive Concept 16, wherein the engaged lengthwise portion of the first guidewire is at least one of welded, soldered, fused, and heat-welded to a radial wall of the tubular member such that no more than an angular minority of a circumference of the lengthwise portion is in fixed contact with the radial wall.

Inventive Concept 25. The method of any one of Inventive Concepts 16 to 24, wherein the engaged lengthwise portion of the first guidewire has a diameter less than a diameter of a different portion of the first guidewire that is not engaged with the tubular member.

Inventive Concept 26. The method of any one of Inventive Concepts 16 to 25, wherein the engaged lengthwise portion of the first guidewire includes a distal end of the first guidewire.

Inventive Concept 27. The method of any one of Inventive Concepts 16 to 26, wherein the engaged lengthwise portion of the first guidewire is engaged with at least a lengthwise majority of the tubular member.

Inventive Concept 28. The method of Inventive Concept 27, wherein the engaged lengthwise portion of the first guidewire is engaged with substantially all of the tubular member.

Inventive Concept 29. The method of any one of Inventive Concepts 16 to 28, wherein the engaged lengthwise portion of the first guidewire is not indestructibly separable from the tubular member.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A medical system for coupling to a previously-introduced guidewire during an intracorporeal procedure, the medical system comprising:
a. a supplemental guidewire; and
b. a single-lumen tubular member comprising a radial wall fixedly engaged to a lengthwise portion of the supplemental guidewire, the radial wall defining a passageway sized for slidable passage therethrough of the previously introduced guidewire,
wherein the tubular portion and the engaged lengthwise portion of the supplemental guidewire are not engaged coaxially.

2. The medical system of claim 1, wherein the tubular portion and the supplemental guidewire are not coaxial.

3. The medical system of either one of claims 1 or 2, wherein a central longitudinal axis of the supplemental guidewire is parallel to a central longitudinal axis of the tubular member.

4. The medical system of any one of the preceding claims, wherein a central longitudinal axis of the supplemental guidewire is laterally displaced from a central longitudinal axis of the tubular member.

5. The medical system of any one of the preceding claims, wherein a central longitudinal axis of the engaged lengthwise portion of the supplemental guidewire is parallel to and laterally displaced from a central longitudinal axis of the tubular member.

6. The medical system of any one of the preceding claims, wherein the engaged lengthwise portion of the supplemental guidewire is at least partly embedded longitudinally in the radial wall of the tubular member.

7. The medical system of any one of the preceding claims, wherein the fixed engaging of the tubular member with the lengthwise portion includes at least one of molding, overmolding, extruding, welding, heat-welding, gluing, soldering, and fusing.

8. The medical system of any one of the preceding claims, wherein the tubular member is molded and/or extruded to at least partly embed, in said radial wall, the lengthwise portion of the supplemental guidewire.

9. The medical system of any one of the preceding claims, wherein an angular majority of a circumference of the engaged lengthwise portion is in fixed contact with the radial wall.

10. The medical system of any one of the preceding claims, wherein at most an angular minority of a circumference of the engaged lengthwise portion is in fixed contact with the radial wall.

11. The medical system of any one of the preceding claims, wherein the engaged lengthwise portion of the supplemental guidewire includes a distal end of the supplemental guidewire.

12. The medical system of any one of the preceding claims, wherein the engaged lengthwise portion of the supplemental guidewire is not indestructibly separable from the tubular member.

13. A method of using multiple guidewires, the method comprising:
a. providing the medical system of any one of the preceding claims;
b. locating a proximal end of the previously-introduced guidewire;
c. slidably passing the tubular member of the medical system over the proximal end of the previously-introduced guidewire so that said proximal end enters a distal opening of the tubular member and exits a proximal opening thereof; and
d. distally advancing the medical system over the previously-introduced guidewire.

14. A method of coupling to a previously-introduced guidewire during an intracorporeal procedure, the method comprising:
a. providing a medical system comprising a supplemental guidewire and a single-lumen tubular member comprising a radial wall fixedly and non-coaxially engaged with a lengthwise portion of the supplemental guidewire;
b. locating a proximal end of a previously-introduced guidewire;
c. slidably passing the tubular member of the medical system over the proximal end of the previously-introduced guidewire so that said proximal end enters a distal opening of the tubular member and exits a proximal opening thereof; and
d. distally advancing the medical system over the previously-introduced guidewire.

15. The method of claim 14, wherein the tubular portion and the supplemental guidewire are not coaxial.

16. The method of either one of claims 14 or 15, wherein a central longitudinal axis of the supplemental guidewire is parallel to a central longitudinal axis of the tubular member.

17. The method of any one of claims 14 to 16, wherein a central longitudinal axis of the supplemental guidewire is laterally displaced from a central longitudinal axis of the tubular member.

18. The method of any one of claims 14 to 17, wherein a central longitudinal axis of the engaged lengthwise portion of the supplemental guidewire is parallel to and laterally displaced from a central longitudinal axis of the tubular member.

19. The method of any one of claims 14 to 18, wherein the engaged lengthwise portion of the supplemental guidewire is at least partly embedded longitudinally in a radial wall of the tubular member.

20. The method of any one of claims 14 to 19, wherein the tubular member is molded and/or extruded to at least partly embed, in said radial wall, the lengthwise portion of the supplemental guidewire.

21. The method of any one of claims 14 to 20, wherein an angular majority of a circumference of the lengthwise portion is in fixed contact with the radial wall.

22. The method of any one of claims 14 to 21, wherein the engaged lengthwise portion of the supplemental guidewire includes a distal end of the supplemental guidewire.

23. The method of any one of claims 14 to 22, wherein the engaged lengthwise portion of the supplemental guidewire is not indestructible separable from the tubular member.
